(19) United States
(12) Patent Application Publication
(10) Pub. No.: US 2022/0107942 A1
(43) Pub. Date: Apr. 7, 2022

(54) SYSTEMS AND METHODS FOR CENTRALIZED MACHINE LEARNING-BASED IMPACT ATTRIBUTION

(71) Applicant: b8ta, Inc., San Francisco, CA (US)

(72) Inventors: Vibhu Norby, Mountain View, CA (US); Joseph Noon, San Ramon, CA (US)

(21) Appl. No.: 17/479,599

(22) Filed: Sep. 20, 2021

Related U.S. Application Data
(60) Provisional application No. 63/081,300, filed on Sep. 21, 2020.

(51) Int. Cl. G06F 16/248; G06F 16/28

(52) U.S. Cl. CPC G06F 16/248; G06F 16/28

(57) ABSTRACT

Obtaining historical data for transactions, the historical data including a number of transactions within the first geographic area and respective shipping locations of the transactions within the first geographic area. Selecting a first respective shipping location of a first transaction within the first geographic area. Selecting a second geographic area based on the first respective shipping location, the first respective shipping location being within the second geographic area. Determining an impact of a physical store based on a set of characteristic values and a subset of the historical data corresponding to the second geographic area. Generating one or more map visualizations based on the impact determination. Presenting the one or more map visualizations. Triggering, in response to generating the map visualization, one or more actions. Generating, subsequent to the one or more actions being triggered and/or performed, an updated impact determination. Presenting the one or more updated map visualizations.

### BRIEF DESCRIPTION OF THE DRAWINGS

FIG. 1 is an image of an example map visualization.

FIG. 2 is a diagram of an example of a system for centralized machine learning-based impact attribution.

FIG. 3 is a diagram of an example centralized machine learning-based impact attribution system.

FIG. 4 is a flowchart of an example of a method of centralized machine learning-based impact attribution associated with a disease (e.g., COVID-19).

FIG. 5 is a flowchart of an example of a method of centralized machine learning-based impact attribution associated with transactions.

### DETAILED DESCRIPTION

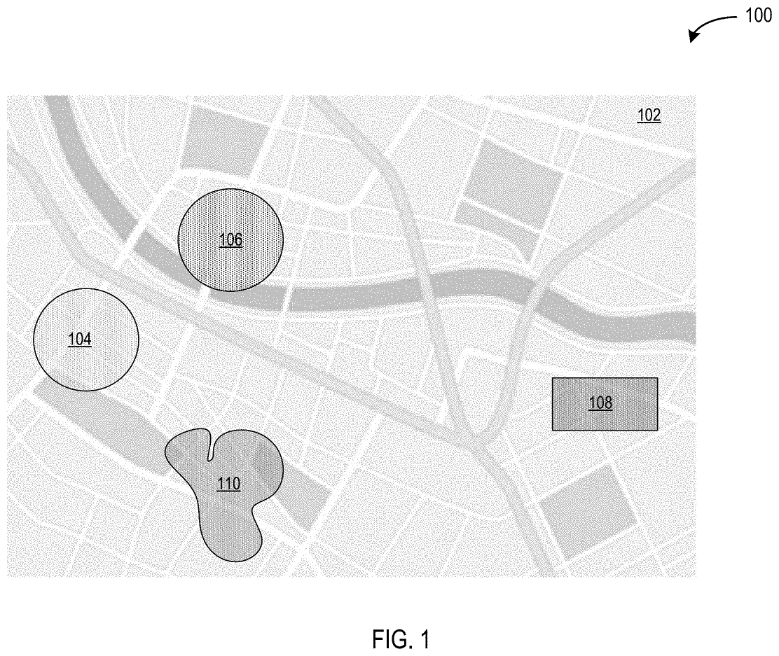

FIG. 1 is an image 100 of an example map visualization 102. In a. specific implementation, the map visualization 102 can be generated by one or more of the systems and methods described herein. As used in this paper, map visualizations can include heatmaps, tree maps, and/or the Map visualizations can indicate impact as colors, patterns, shapes, and/or the like. In the example of FIG. 1, the map visualization 102 is a heatmap. The map visualization 102 is associated with a disease (e.g., COVID-19) and indicates the impact that infected individuals have within particular portions of the geographic area. Impact can include a number of disease transmissions or expected disease transmissions, a number of disease-related deaths or expected disease-related deaths, effects on hospitals or expected effects on hospitals (e.g., number of used or available beds in the hospital), and/or the like. The map visualization 102 includes map visualization elements 104-110 overlaid on a geographic map.

The map visualization element 104 indicates an impact of a first infected individual within a particular radius, as shown by the boundaries of the map visualization element 104. Although a circle and radius are used for the map visualization element 104, other shapes, dimensions and/or features can be used for other map visualization elements (e.g., as shown in map visualization elements 108 and 110). In the example of FIG. 1, the map visualization element 104 represents the first infected individual at a center of the map visualization element 104, and indicates the impact that the first infected individual has within the area covered by the map visualization element 104.

The map visualization element 106 indicates an impact of a second infected individual. As shown by a darker shading, the second infected individual has a higher impact relative to the first infected individual. This may occur due to one or more different characteristics of the respective infected individuals or the respective geographic areas. For example, the geographic area covered by the map visualization element 104 may be associated with a local ordinance requiring individuals to wear a mask, while the geographic area covered by the map visualization element 106 may not be associated with such a local ordinance.

The map visualization elements 108 and 110 indicate, respectively, the impact of a third infected individual and a fourth infected individual.

The map visualization 102 can also be used to attribute impact to infected individuals (e.g., the infected individual associated with map visualization element 104 has caused, or will cause, 5 disease transmissions and caused, or will cause, 1 used hospital bed). The map visualization 102 can also be used to trigger one or more actions. For example, if an impact is greater than an impact threshold, one or more notifications can be sent (e.g., notifying public health officials) or suggest local ordinance modifications (e.g., enact a facemask policy). Conversely, if an impact is below an impact threshold, one or more notifications can also be sent or different local ordinance modifications can be suggested (e.g., remove a facemask policy).

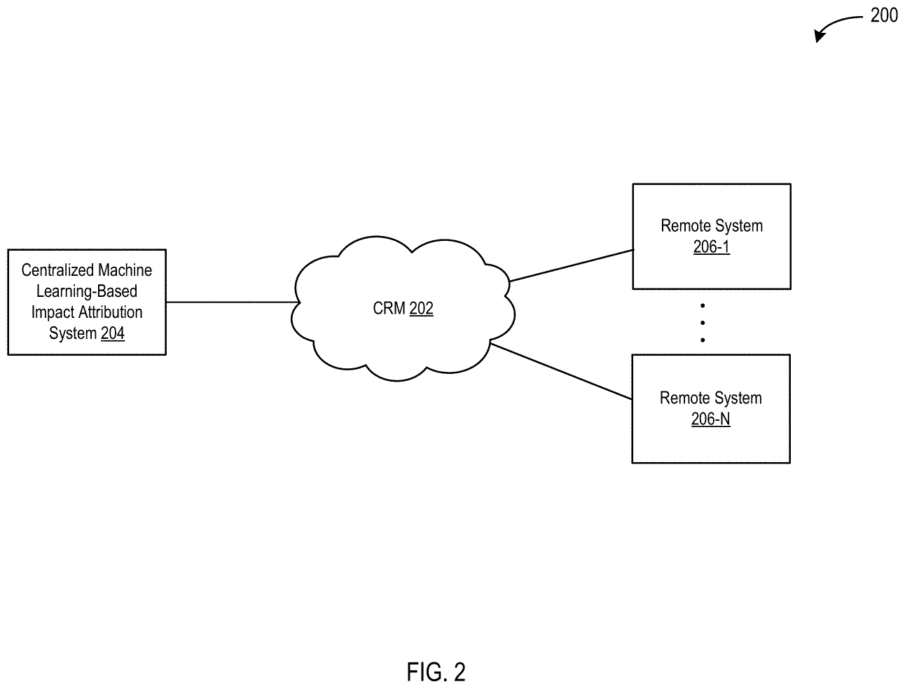

FIG. 2 is a diagram 200 of an example of a system for centralized machine learning-based impact attribution. The diagram 200 includes a computer-readable medium (CRM) 202, a centralized machine learning-based impact attribution system 104 coupled to the CRM 102, and remote systems 106-1 to 106-N (individually, the remote system 106, collectively, the remote systems 106) coupled to the CRM 102.

The CRM 102 in intended to represent a computer system or network of computer systems. A “computer system,” as used herein, may include or be implemented as a specific purpose computer system for carrying out the functionalities described in this paper. In general, a computer system will include a processor, memory, non-volatile storage, and an interface. A typical computer system will usually include at least a processor, memory, and a device (e.g., a bus) coupling the memory to the processor. The processor can be, for example, a general-purpose central processing unit (CPU), such as a microprocessor, or a special-purpose processor, such as a microcontroller.

Memory of a computer system includes, by way of example but not limitation, random access memory (RAM), such as dynamic RAM (DRAM) and static RAM (SRAM). The memory can be local, remote, or distributed. Non-volatile storage is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. During execution of software, some of this data is often written, by a direct memory access process, into memory by way of a bus coupled to non-volatile storage. Non-volatile storage can be local, remote, or distributed, but is optional because systems can be created with all applicable data available in memory.

Software in a computer system is typically stored in non-volatile storage. Indeed, for large programs, it may not even be possible to store the entire program in memory. For software to run, if necessary, it is moved to a computer-readable location appropriate for processing, and for illustrative purposes in this paper, that location is referred to as memory. Even when software is moved to memory for execution, a processor will typically make use of hardware registers to store values associated with the software, and a local cache that, ideally, serves to speed up execution. As used herein, a software program is assumed to be stored at an applicable known or convenient location (from non-volatile storage to hardware registers) when the software program is referred to as “implemented in a computer-readable storage medium.” A processor is considered “configured to execute a program” when at least one value associated with the program is stored in a register readable by the processor.

In one example of operation, a computer system can be controlled by operating system software, which is a software program that includes a file management system, such as a disk operating system. One example of operating system software with associated file management system software is the family of operating systems known as Windows from Microsoft Corporation of Redmond. Wash., and their associated file management systems. Another example of operating system software with its associated file management system software is the Linux operating system and its associated file management system. The file management system is typically stored in the non-volatile storage and causes the processor to execute the various acts required by the operating system to input and output data and to store data in the memory, including storing files on the non-volatile storage.

The bus of a computer system can couple a processor to an interface. Interfaces facilitate the coupling of devices and computer systems. Interfaces can be for input and/or output (I/O) devices, modems, or networks. I/O devices can include, by way of example but not limitation, a keyboard, a mouse or other pointing device, disk drives, printers, a scanner, and other I/O devices, including a display device. Display devices can include, by way of example but not limitation, a cathode ray tube (CRT), liquid crystal display (LCD), or some other applicable known or convenient display device. Modems can include, by way of example but not limitation, an analog modem, an IDSN modem, a cable modem, and other modems. Network interfaces can include, by way of example but not limitation, a token ring interface, a satellite transmission interface (e.g. “direct PC”), or other network interface for coupling a first computer system to a second computer system. An interface can be considered part of a device or computer system.

Computer systems can be compatible with or implemented as part of or through a cloud-based computing system. As used in this paper, a cloud-based computing system is a system that provides virtualized computing resources, software and/or information to client devices. The computing resources, software and/or information can be virtualized by maintaining centralized services and resources that the edge devices can access over a communication interface, such as a network. “Cloud” may be a marketing term and for the purposes of this paper can include any of the networks described herein. The cloud-based computing system can involve a subscription for services or use a utility pricing model. Users can access the protocols of the cloud-based computing system through a web browser or other container application located on their client device.

A computer system can be implemented as an engine, as part of an engine, or through multiple engines. As used in this paper, an engine includes at least two components: 1) a dedicated or shared processor or a portion thereof; 2) hardware, firmware, and/or software modules executed by the processor. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors, or a first engine and a second engine can share one or more processors with one another or other engines. Depending upon implementation-specific or other considerations, an engine can be centralized, or its functionality distributed. An engine can include hardware, firmware, or software embodied in a computer-readable medium for execution by the processor. The processor transforms data into new data using implemented data structures and methods, such as is described with reference to the figures in this paper.

The engines described in this paper, or the engines through which the systems and devices described in this paper can be implemented, can be cloud-based engines. As used in this paper, a cloud-based engine is an engine that can run applications and/or functionalities using a cloud-based computing system. All or portions of the applications and/or functionalities can be distributed across multiple computing devices and need not be restricted to only one computing device. In some embodiments, the cloud-based engines can execute functionalities and/or modules that end users access through a web browser or container application without having the functionalities and/or modules installed locally on the end-users' computing devices.

As used in this paper, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a general- or specific-purpose machine, in firmware, in hardware, in a combination thereof, or in an applicable known or convenient device or system. Datastore-associated components, such as database interfaces, can be considered “part of” a datastore, part of sonic other system component, or a combination thereof, though the physical location and other characteristics of datastore-associated components is not critical for an understanding of the techniques described in this paper.

Datastores can include data structures. As used in this paper, a data structure is associated with a way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described in this paper, can be cloud-based datastores. A cloud based datastore is a datastore that is compatible with cloud-based computing systems and engines.

Assuming a CRM includes a network, the network can be an applicable communications network, s as the Internet or an infrastructure network. The term “Internet” as used in this paper refers to a network of networks that use certain protocols, such as the TCP/IP protocol, and possibly other protocols, such as the hypertext transfer protocol (HTTP) for hypertext markup language (HTML) documents that make up the World Wide Web (“the web”). More generally, a network can include, for example, a wide area network (WAN), metropolitan area network (MAN), campus area network (CAN), or local area network (LAN), but the network could at least theoretically be of an applicable size or characterized in some other fashion (e.g., personal area network (PAN) or home area network (HAN), to name a couple of alternatives). Networks can include enterprise private networks and virtual private networks (collectively, private networks). As the name suggests, private networks are under the control of a single entity. Private networks can include a head office and optional regional offices (collectively, offices). Many offices enable remote users to connect to the private network offices via some other network, such as the Internet.

The centralized machine learning-based impact attribution system 204 can function to determine an impact and attribute an impact, For example, the centralized machine learning-based impact attribution system 204 can determine an impact that infected individuals (e.g., infected with COVID-19) have within various geographic areas, generate corresponding map visualizations, and/or trigger, one or more actions. Functionality of centralized machine learning-based impact attribution system 204 can be performed by one or more servers (e.g., a cloud-based server) and/or other computing devices.

The remote systems 206 can function to collect, transmit, access, and/or modify data (e.g., location data, transaction data). Functionality of the remote systems can be performed by one or more mobile devices (e.g., smartphones), servers (e.g., online retail system), and/or the like.

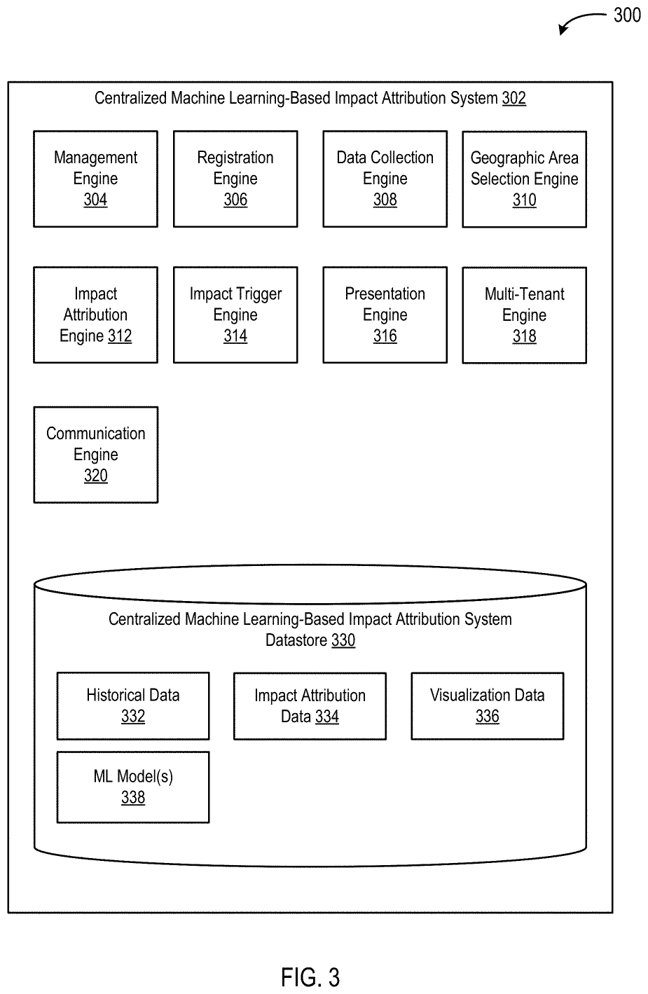

FIG. 3 is a diagram 300 of an example centralized machine learning-based impact attribution system 302. The diagram 300 includes a management engine 304, a registration engine 306, a data collection engine 308, a geographic area selection engine 310, an impact attribution engine 312, an impact trigger engine 314, a presentation engine 316, a multi-tenant engine 318, and a communication engine 320.

The management engine 304 is intended to represent an engine that manages (e.g., create, read, update, delete, or otherwise access) historical data 332, impact attribution data 334, visualization data 336, and/or machine learning model(s) 338. The management engine 204 can perform any of these operations manually (e.g., by a user interacting with a GUI) and/or automatically (e.g., triggered by one or more of the engines 306-320). Like other engines described herein, some or all the functionality of the management engine 304 can be included in and/or cooperate with one or more other engines (e.g., engines 306-320).

The registration engine 306 is intended to represent an engine that registers systems, data, and/or entities. The registration engine 306 can perform registration manually aid/or automatically. For example, the registration engine 306 can perform registration via one or more application programing interfaces (APIs) of an associated service Shopify). The registration can include generating login credentials (e.g., username, password), and facilitate data onboarding. For example, the registration engine 306 can trigger one or more other engines to onboard data (e.g., historical data 332).

The data collection engine 308 is intended to represent an engine that collects data. For example, the data collection engine 308 can onboard data manually and/or automatically. For example, the data collection engine 308 can onboard data via one or more APIs as part of a registration process.

In a specific implementation, the data collection engine 308 collects data in different formats (e.g., from remote systems). The data collection engine 308 can normalize the different formats to one or more normalized formats in order for the centralized machine learning-based impact attribution system 302 to operate on the data. For example, one or more of the other engines can operate on normalized historical data rather than an original format of the historical data as obtained (e.g., from remote systems). Accordingly, the centralized machine learning-based impact attribution system 302 can function regardless of the original format of the collected data. This can also allow faster onboarding of data.

The geographic area selection engine 310 is intended to represent an engine that selects geographic areas either manually or automatically. The geographic area selection engine 310 can select geographic areas based on one or more attributes (e.g., radius). The attributes can be predetermined (e.g., 10-mile radius) or variable. For example, the attribute can initially be set to a 10-mile radius and modified to a lower or higher radius. Selected geographic areas can have different shapes (e.g., circle, rectangle, and/or the like).

The impact attribution engine 312 is intended to represent an engine that determines and attributes impact based on machine learning techniques. In a specific implementation, the impact attribution engine 312 uses a machine learning model 338 (e.g., a random forests model) and a set of one or more weighted characteristics to determine or predict an impact, and then attribute that impact (e.g., to an entity or a location). For example, the weighted characteristics can include, or be based on, demographic information, psychographic information, and/or the like. In a specific implementation, the impact attribution engine 312 can set a baseline value and determine an impact value relative to the baseline value.

The impact trigger engine 314 is intended to represent an engine that triggers or suggests one or more actions. The impact trigger engine 314 can trigger actions in response to satisfaction of one or more conditions or thresholds. In a specific implementation, the impact trigger engine 314 can trigger actions based on one or more map visualizations and/or one or more conditions or thresholds.

The presentation engine 316 is intended to represent an engine that presents audio, visual, and/or haptic information. In some embodiments, the presentation engine 316 generates a graphical user interface including one or more map visualizations.

The multi-tenant engine 318 is intended to represent an engine that can securely store data of different tenants. In a specific implementation, data of different tenants can be stored and accessed by the various tenants as if they were the only tenant. For example, historical data 332 of a first tenant can be securely walled-off from historical data 332 of another tenant. Accordingly, the multi-tenant engine 318 can facilitate the centralization of impact determination and attribution, and reduce the amount of information that needs to be transmitted, thereby reducing bandwidth requirements, data transmission payloads, and/or the like.

The communication engine 320 is intended to represent an engine that sends requests, transmits and, receives communications, and/or otherwise provides communication with one or more of the systems, engines, devices and/or datastores described herein. In a specific implementation, the communication engine 320 can function to encrypt and decrypt communications. The communication engine 320 can function to send requests to and receive data from one or more systems through a network or a portion of a network (e.g., communications network 108, communications network 608). In a specific implementation, the communication engine 220 can send requests and receive data through a connection, all or a portion of which can be a wireless connection. The communication engine 220 can request and receive messages, and/or other communications from associated systems and/or engines. Communications can be stored in the centralized machine learning-based impact attribution system datastore 330.

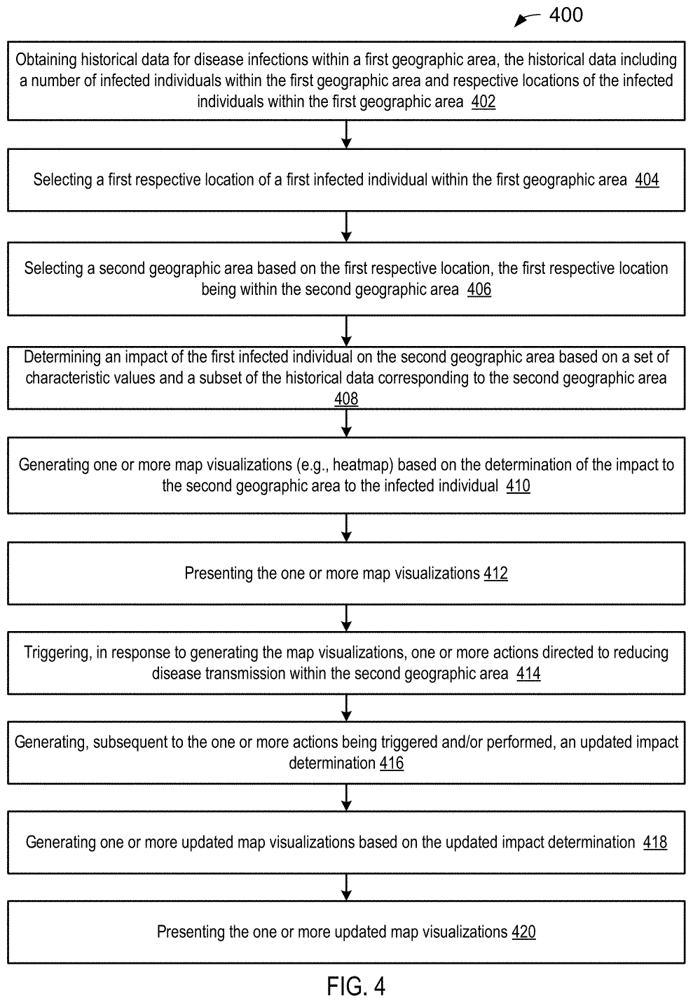

FIG. 4 is a flowchart 400 of an example of a method of centralized machine learning-based impact attribution associated with a disease (e.g., COVID-19). In this and other flowcharts and/or sequence diagrams, the flowchart illustrates by way of example a sequence of modules. It should be understood the modules can be reorganized for parallel execution, or reordered, as applicable. Moreover, some modules that could have been included may have been removed to avoid providing too much information for the sake of clarity and some modules that were included could be removed, but may have been included for the sake of illustrative clarity.

In module 402, a centralized machine learning-based impact attribution system obtains historical data for disease infections within a first geographic area. For example, the first geographic area can be a particular city or town, or other geographic area. The historical data can include a number of infected individuals within the first geographic area and respective locations of the infected individuals within the first geographic area. In a specific implementation, a geographic area selection engine selects the first geographic area, and a data collection engine obtains the historical information from one or more remote systems. For example, the one or more remote system can be mobile devices of individuals (e.g., infected individuals), and the locations can be based on location information provided by the mobile devices.

In a specific implementation, the data collection engine obtains the historical data in different formats, and normalizes the historical data to a normalized format, and subsequent modules of the flowchart 400 operate on the normalized historical data.

In module 404, the centralized machine learning-based impact attribution system selects a first respective location of a first infected individual within the first geographic area.

In module 406, the centralized machine learning-based impact attribution system selects a second geographic area based on the first respective location. The first respective location can be within the second geographic area. For example, the second geographic area can be circular area having a 10-mile radius from the first respective location. As noted elsewhere herein, the second. geographic area may have a different shape and/or size.

In module 408, the centralized machine learning-based impact attribution system determines an impact of the first infected individual on the second geographic area based on a set of characteristic values and a subset of the historical data corresponding to the second geographic area. For example, the set of characteristic values, which can be weighted, can include characteristics of the disease transmission probabilities, incubation periods), characteristics of the first infected individual (e.g., demographic information, psychographic information), and/or characteristics of the second geographic area (e.g., local ordinances regarding wearing facemasks). An impact attribution engine can use the set of characteristics, or portion thereof, as input to a machine learning model (e.g., random forests model), and use the output of the model to determine the impact and then attribute that impact to the first infected individual.

In a specific implementation, the determined impact is relative to a baseline value. For example, a baseline value can be that an individual infects 2.5 people. The impact attribution engine can determine that the first infected individual has infected, or will infect, 4 people. The impact attribution engine can determine the impact relative to the difference (e.g., difference between 4 and 2.5). For example, the relative impact can be the difference (e.g., 1.5), or a portion thereof (e.g., 10% of 1.5). The portion of the difference can be selected or determined automatically based on machine learning. For example, the initial attribution can be set to 10% of the difference, but then adjusted by a machine learning model using subsequently obtained historical data which indicates that 10% is either too high or too low of an attribution. Accordingly, the attributed impact can become more accurate over time.

In module 410, the centralized machine learning-based impact attribution system generates one or more map visualizations (e.g., heatmap) based on the determination of the impact to the second geographic area to the infected individual. An example map visualization that can be generated is shown in FIG. 1.

In module 412, the centralized machine learning-based impact attribution system presents the one or more map visualizations.

In module 414, the centralized machine learning-based impact attribution system triggers, in response to generating the map visualizations, one or more actions directed to reducing disease transmission within the second geographic area. For example, actions can include adjusting ordinances or zoning regulations (e.g., opening/closing restaurants, adding/removing facemask policies. In a specific implementation, the actions adjust or modify values of the set of characteristics.

In module 416, the centralized machine learning-based impact attribution system generates, subsequent to the one or more actions being triggered and/or performed, an updated impact determination. The updated impact determination can be based on updated characteristic values that may have been updated by the one or more actions) that are input in the machine learning model.

In module 418, the centralized machine learning-based impact attribution system generates one or more updated map visualizations based on the updated impact determination. For example, the initial map visualization may have been based on the second geographic area being associated with a local ordinance mandating individuals wear facemasks, and the updated map visualization may have been generated after that local ordinance is removed.

In module 420, the centralized machine learning-based impact attribution system presents the one or more updated map visualizations.

In a specific implementation, any number of the modules can be repeated (e.g., iterated) to achieve more accurate or current impact determinations, attributions, and/or map visualizations.

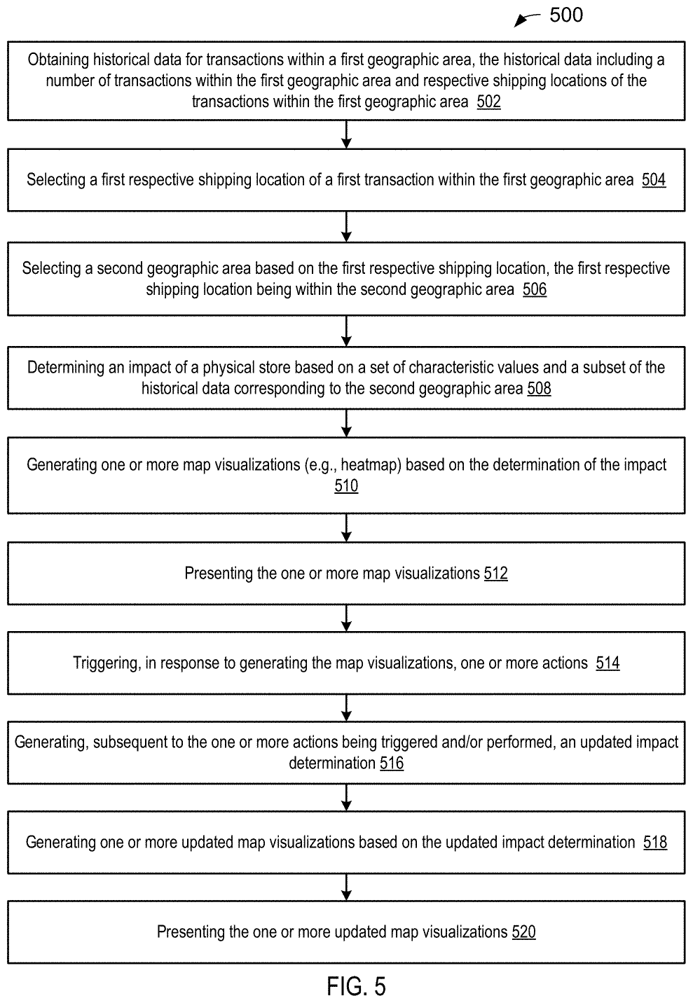

FIG. 5 is a flowchart 500 of an example of method of centralized machine learning-based impact attribution associated with transaction data (e.g., sales transaction data). In this and other flowcharts and/or sequence diagrams, the flowchart illustrates by way of example a sequence of modules. It should be understood the modules can be reorganized for parallel execution, or reordered, as applicable. Moreover, some modules that could have been included may have been removed to avoid providing too much information for the sake of clarity and some modules that were included could be removed, but may have been included for the sake of illustrative clarity.

In module 502, a centralized machine learning-based impact attribution system obtains historical data for transactions within a first geographic area. For example, the first geographic area can be a particular city or town, or other geographic area. The historical data can include a number of sales transactions for one or more items (e.g., physical store sales transactions, online store sales transactions) and amounts of the sales transactions within the first geographic area and respective shipping locations (e.g., a physical address) of a receiving entity.

In a specific implementation, a geographic area selection engine selects the first geographic area, and a data collection engine obtains the historical information from one or more remote systems. For example, the one or more remote systems can include a retailer system and a brand system. The retailer system can operate the centralized machine learning-based impact attribution system, and the brand system can be an online/e-commerce system that completed the transactions. A data collection engine can onboard the historical data as part of a registration process, and a multi-tenant engine can facilitate secure storage and access of the historical data.

In a specific implementation, the data, collection engine obtains the historical data, in different formats, and normalizes the historical data to a normalized format, and subsequent modules of the flowchart 600 operate on the normalized historical data.

In module 504, the centralized machine learning-based impact attribution system selects a first respective shipping location of a first transaction within the first geographic area.

In module 506, the centralized machine learning-based impact attribution system selects a second geographic area based on the first respective location. The first respective location can be within the second geographic area. For example, the second geographic area can be a circular area having a 10-mile radius from a physical store that sells the same item as the item of the first transaction. In another example, the second geographic area can be a circular area having a 10-mile radius from an anticipated physical store that will sell the same item as the item of the first transaction. As noted elsewhere herein, the second geographic area may have a different shape and/or size.

In module 508, the centralized machine learning-based impact attribution system determines an impact of a physical store based on a set of characteristic values and a subset of the historical data corresponding to the second geographic area. For example, the set of characteristic values, which can be weighted, can include characteristics of the first transaction (e.g., sale amount, whether shipping location is within the second geographic area or outside the second geographic area), characteristics of the receiving entity (e.g., demographic information, psychographic information), and/or characteristics of the second geographic area. Characteristics of the second geographic area can include whether the second geographic area has a corresponding physical store (e.g., a physical store that sells the item) or has previously had a corresponding physical store. An impact attribution engine can use the set of characteristics, or portion thereof, as input to a machine learning model (e.g., random forests model), and use the output of the model to determine the impact and then attribute that impact to the particular physical store.

In a specific implementation, the determined impact is relative to a baseline value. For example, a baseline value can be that a brand sells online $1,000 of a particular item per month (e.g., as indicated by the historical data over a period of time) when there is no physical store in that area selling that item. After a physical store is added to that area, the brand sells $1,500 of that particular item per month. The impact attribution engine can determine the impact relative to the difference (e.g., difference between $1,500 and $1,000). For example, the relative impact can be the difference (e.g., $500), or a portion thereof (e.g., 10% of $500). The portion of the difference can be selected or determined automatically based on machine learning. Accordingly, the attributed impact can become more accurate over time.

In a specific implementation, the centralized machine learning-based impact attribution system predicts how adding a physical store or other characteristics will impact online transactions. For example, the centralized machine learning-based impact attribution system can use the historical data as well as anonymized data of other brands as input to a machine learning model to predict whether online sales will increase and by how much. This can facilitate the determination (e.g., automatically or manually) by the system of a portion of the difference to attribute (e.g., 10%, 5%, 15%). For example, the centralized machine learning-based impact attribution system can use anonymized data of similar transactions of other brands (e.g., transactions for items in a same category or sold by other brands in a same category) as a portion of the characteristic values input into the machine learning model. Predicting impact can be similarly applied to other use-cases (e.g., disease use-cases).

In module 510, the centralized machine learning-based impact attribution system generates one or more map visualizations (e.g., heatmap) based on the determination of the impact.

In module 512, the centralized machine learning-based impact attribution system presents the one or more map visualizations.

In module 514, the centralized machine learning-based impact attribution system triggers, in response to generating the map visualizations, one or more actions. For example, actions can include invoicing a brand system based on attribution amount and sales amounts. Invoicing can also occur manually or in response to other triggers (e.g., a time-based trigger, such as a monthly invoice).

In module 516, the centralized machine learning-based impact attribution system generates, subsequent to the one or more actions being triggered and/or performed, an updated impact determination. The updated impact determination can be based on updated characteristic values (e.g., that may have been updated by the one or more actions) that are input in the machine learning model.

In module 518, the centralized machine learning-based impact attribution system generates one or more updated map visualizations based on the updated impact determination. For example, the initial map visualization may have been based on one store located in the second geographic area, and the updated map visualization may have been generated after a second store has been added to the second geographic location.

In module 520, the centralized machine learning-based impact attribution system presents the one or more updated map visualizations.

In a specific implementation, any number of the modules can be repeated (e.g., iterated) to achieve more accurate or current impact determinations, attributions, and/or map visualizations.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

The present invention(s) are described above with reference to example embodiments. It will be apparent to those skilled in the art that various modifications may be made and other embodiments may be used without departing from the broader scope of the present invention(s). Therefore, these and other variations upon the example embodiment are intended to be covered by the present invention(s).

1. A method comprising;
Obtaining historical data for transaction within a first geographic area, the historical data. including a number of transactions within the first geographic area and respective shipping locations of the transactions within the first geographic area;
Selecting a first respective shipping location of a first transaction within the first geographic area;
Selecting a second geographic area based on the first respective shipping location, the first respective shipping location being within the second geographic area;
Determining an impact of a physical store based on a set of characteristic values and a subset of the historical data corresponding to the second geographic area;
Generating one or more map visualizations (e.g., heatmap) based on the determination of the impact;
Presenting the one or more map visualizations;
Triggering, in response to generating the map visualization, one or more actions;
Generating, subsequent to the one or more actions being triggered an/or performed, an updated impact determination;
Presenting the one or more updated map visualizations.
